(19) United States
(12) Patent Application Publication
(10) Pub. No.: US 2005/0043548 A1
(43) Pub. Date: Feb. 24, 2005

(54) Automated monitoring and control system for networked communications

(76) Inventor: Joseph Cates, Glendale, CA (US)

Correspondence Address: DARBY & DARBY P.C., P. O. BOX 5257, NEW YORK, NY, 10150-5257, US

(21) Appl. No.: 10/848,556

(22) Filed: May 17, 2004

Related U.S. Application Data
(63) Continuation of application No. 10/646,465, filed on Aug. 22, 2003, now abandoned.

(51) Int. Cl. C07D 337/16

(52) U.S. Cl. 549/12

(57) ABSTRACT

A monitoring station is used to monitor a computer network for illegal or undesirable content. The monitoring station conducts searches based on a set of criteria and optionally downloads and examines suspected files. Once the monitoring station identifies illegal or undesirable content, it sends an electronic notice to a control station. The control station derives a enforcement order from the electronic notice based on enforcement rules. The control station sends the enforcement order to a traffic controller. The traffic controller executes the enforcement rule by limiting the access of a network node. In a further embodiment multiple monitoring stations, control stations and traffic controllers may be used.

### FIELD OF THE INVENTION

This Invention relates generally to networked computer systems and more specifically to tools for monitoring and controlling communications over networked computer systems based on the content being transmitted.

### BACKGROUND OF THE INVENTION

The wide adoption of the Internet has resulted in the lowering of the costs of distributing information in some environments to almost trivial levels. This has had many positive effects, but unfortunately some negative ones as well. Namely, illegal or otherwise undesirable information exchange has proliferated. Such exchange includes transfers of copyrighted material in violation of copyright, and transfers of harmful programs such as viruses, trojan horses or other destructive programs.

Currently, exchange of illegal or undesirable content is controlled mostly by the Internet Service Provider(s) (ISPs) that provide service to one or more of the offending parties. Most ISPs, however, do not take it upon themselves to be an active monitor of undesirable content. They usually rely on affected parties, such as copyright holders, to alert them to allegedly undesirable content. Once such an alert is made, an ISP usually determines whether the content should be restricted (i.e., taken off a webpage, or a server) and whether the user trafficking in the content should be reprimanded. Usually an ISP makes such a determination based on legal requirements, such as, for example, the Digital Millennium Copyright Act (DMCA), the ISP's own technical risks and costs, and the ISP's terms of use. The ISP's terms of use is usually in the form of an agreement between an ISP and all its clients governing the use of the ISP's services.

Currently, the above described process is performed manually. Often, an affected party must take legal action in order to impress upon the ISP the gravity of its request. Similarly, the ISP may decide to make a manual determination of the affected party's request, in order to avoid alienating its customers on the one hand, and to avoid the legal and moral ramifications of its customer's illegal behavior on the other hand. This results in unnecessarily high costs for both the affected party and the ISP. Thus, while the cost of undesirable communication exchange is extremely low, the cost of policing such exchange remains high. In other words, the exchange of undesirable information benefits from all the efficiencies of modern computing technology, while policing it does not, because policing still requires significant human involvement.

Distribution of undesirable content can happen in various ways over computer networks. But there are several methods that are used very often for such purposes. One is peer to peer networking. Peer to peer networking is an efficient method of exchanging information in a decentralized way. It is usually performed over the Internet. It characterizes the exchange of information between two computers, which both belong to individual users, the exchange being accomplished without a central server. Peer to peer communication holds potential for de-centralized computer networking, but unfortunately it is often used to improperly exchange copyrighted content.

Another method of improperly exchanging information utilizes a file transfer protocol (FTP) server. There are many FTP servers on the Internet, with some even dedicated to the improper distribution of copyrighted content.

Other methods of improperly exchanging information include using Internet services such as Usenet, and Internet relay chat (IRC). Both of these Internet services were initially designed for the exchange of plain text information but now any kind of content can be encoded and distributed.

### SUMMARY OF THE INVENTION

The present invention provides for the automation of the policing of the illegal or otherwise undesirable exchange of content.

The present invention automatically attempts to receive content from sources which are likely to provide undesirable content. The present invention then determines if the content is in fact undesirable. If undesirable content is found, the present invention determines a response action based on the nature of the content and executes the response action.

For the purposes of the present invention both the affected party and the ISP must agree to delegate the task of policing for a certain set of illegal content to the system and method of the present invention. Thus, the present invention requires cooperation between the ISP and the affected party. Of course, the present invention may be used when the ISP and the affected party are the same entity. Also the present invention may be used exclusively by the ISP, if the ISP decides to be proactive, and not wait for an affected party to request enforcement.

### BRIEF DESCRIPTION OF THE DRAWINGS

The foregoing and other features of the present invention will be more readily apparent from the following detailed description and drawings of the illustrative embodiments of the invention wherein like reference numbers refer to similar elements and in which:

FIG. 1 is a block diagram of an exemplary embodiment of a monitoring system in accordance with the present invention;

FIG. 2 is a block diagram of an exemplary embodiment of a monitoring system in accordance with the present invention, wherein a single control station is used in conjunction with multiple monitoring stations and multiple traffic controllers; and

FIG. 3 is a block diagram of an exemplary embodiment of a monitoring system in accordance with the present invention wherein multiple monitoring stations, control stations and traffic controllers are used.

### DETAILED DESCRIPTION OF THE PREFERRED EMBODIMENTS

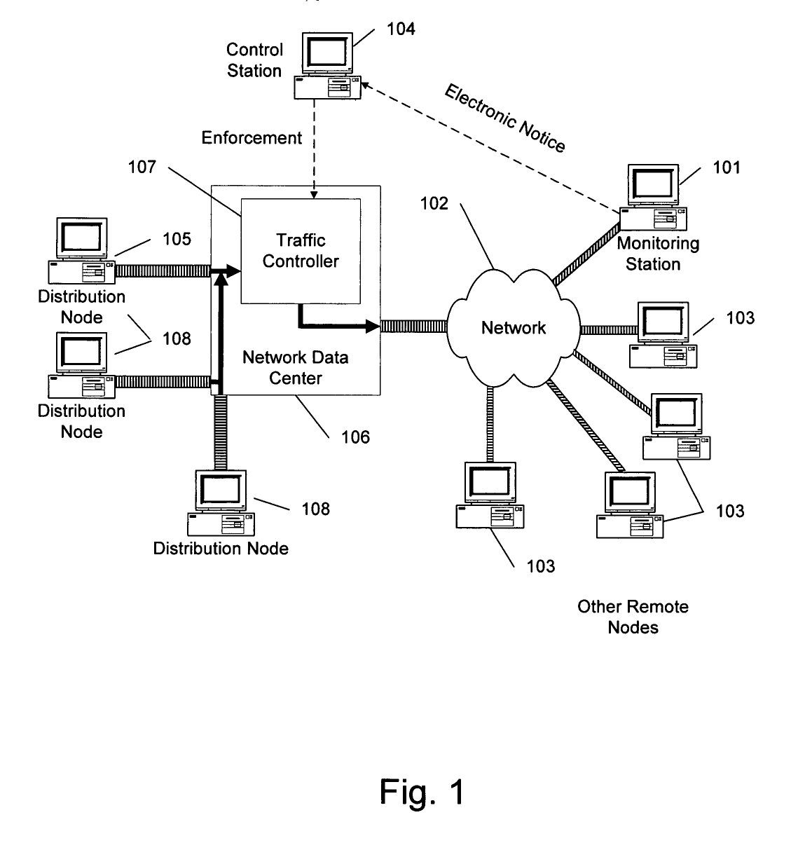

Referring to FIG. 1, a monitoring station 101 is connected to the Internet or another computer network 102. The monitoring station 101 is implemented using a computer or a similar device capable of retrieving content from the network. The monitoring station 101 actively attempts to retrieve content that may be undesirable or the exchange of which may be undesirable, by accessing potential sources of undesirable content.

For this purpose, the monitoring station may read Usenet posts. It may also connect to an IRC server and attempt to retrieve a piece of content from another IRC user. Although IRC is usually a medium for communication in natural language between humans, the monitoring station need not possess the ability to communicate in a natural language in order to retrieve content. This is due to the fact that most of the undesirable content exchanged via IRC is provided by computer programs, running on a user's computer (known as IRC bots). These IRC bots are connected to the IRC as well and listen in for certain commands. A user may issue a command that causes an IRC bot to send a certain file to the user. Thus, the monitoring station need only be able to send some commonly used IRC bot commands in order to retrieve files from the IRC bots. Furthermore, the monitoring station may use some IRC bot commands that list other IRC bot commands, and thus “learn” the commands for a particular bot.

The monitoring station 101 may also connect to known FTP servers, using known or publicly available account names and passwords, and attempt to download content from these sources. The monitoring station may also login to various peer to peer networks, and download content from users by way of such networks. The monitoring station may also connect to other online distribution systems that are likely to be used for distributing illegal content.

The monitoring station 101 is initially configured to search for a set of likely target files. These files may represent movies, audio files, or computer programs, that are known to be distributed in violation of certain copyrights, or computer programs that are known to contain viruses, trojan horses or other malicious software. The monitoring station initially attempts to find the target files by using the target file name, file size, file hash or similar unique identifier, or by other file metadata. Most of the sources of content listed above provide for a way to search for files by one or more of these parameters. Sometimes, the names and other properties of particular files are changed as the files pass through distribution channels, so the monitoring station 101 may have the option to search for matches that are not exact. However, files on the above listed distribution systems may be intentionally, or unintentionally misnamed or mislabeled, especially if enforcement mechanisms are in place. For this reason, the monitoring station 101 does not rely only on the name and properties alone to identify files as undesirable or illegal. If a match based on file name and/or properties is found, the monitoring station 101 may elect to download the file in question. If the download is successful, the monitoring station may further attempt to identify the downloaded file as a target file, by examining its contents. This may be accomplished by examining the file's contents for watermarks or fingerprints.

Fingerprinting is a technology very similar to hashing. A fingerprint is a set of data that is derived from a file by a predefined formula. The formula is such that the chances of two different files having the same fingerprint are very small. In a fingerprinting embodiment, the monitoring station is in possession of the formula as well as a list of fingerprints of one or more target files. The monitoring station then derives the fingerprint of a downloaded file using the formula and compares it to the fingerprints of the target files in the list. A match signifies that the downloaded file is a target file. In order for fingerprinting technology to be effective, the formula should be kept in relative secrecy, so a distributor is not able to change the fingerprint of a file by making minor changes in the file's content.

Watermarking is a technology wherein data is embedded into an existing file, without noticeably changing the end user effect of the file. The end user effect of a file is the effect a file would have on the end user, when used in its intended purpose. Thus adding a watermark to a music file will not noticeably change the audible qualities of the music file. A watermark may signify whether the file it is embedded in is allowed to be distributed over public networks (i.e. whether the file is a target file). In a watermarking embodiment the monitoring station derives the embedded watermark from the downloaded file. By examining the watermark, the monitoring station can determine whether the downloaded file is a target file.

Watermarking may also provide additional information about a file. It may show who the last legal possessor of the file was, and thus determine the person that started the illegal distribution. Or it may designate a file as allowable for distribution on certain distribution networks, but not allowable on others.

In another embodiment, the monitoring station 101 uses both fingerprinting and watermarking.

When downloading a file, the monitoring station 101 stores an address associated with the network node that is distributing the file. In FIG. 1, this node is the distribution node 105. If the distribution node 105 uses the Internet, this identification is usually an IP address. The IP address of a node attempting to distribute unauthorized content is easily obtainable in most distribution systems, because in order for the content to be distributed, a network connection must be created, and knowledge of each party's IP address is necessary for creating a network connection.

Once the monitoring station 101 has identified a downloaded file as a target file, it sends an electronic notice to a control station 104. The electronic notice includes an identification associated with the node that was distributing illegal or undesirable content (distribution node 105 in FIG. 1), and optionally a description of the means of distribution, properties of the content, information on content owner where applicable, and date and time of detection.

Once the control station 104 receives an electronic notice, it determines an enforcement order that is suitable for the electronic notice. An enforcement order is essentially a punishment for illegal or undesirable distribution, which is to be executed against the offending distribution node 105. An enforcement order usually involves limiting a user's network access in a particular way. Table 1 lists some examples of possible enforcement orders.

TABLE 1

| Type of Enforcement Order | Example of Enforcement Order |
|---|---|
| Block specific content incoming | Block specific URL on web server. |
| and/or outgoing for a given time. | |
| Block specific distribution system | Block all FTP traffic from source for 24 |
| traffic incoming and/or outgoing for a | hours. |
| given time. | Block all outgoing SMS traffic from source |
| | for 2 hours. |
| Block all network access incoming | Block all Internet traffic from source for 10 |
| and/or outgoing for a given time. | minutes. |
| | Deactivate source network account for 24 |
| | hours. |
| Assign bandwidth limit or lower | Assign 5 MB Gnutella P2P traffic limit for |
| priority to specific distribution system | source for next 24 hours. |
| traffic incoming and/or outgoing for a | |
| given time. | |
| Assign bandwidth limit or lower | Assign lowest priority to all Internet traffic |
| priority to all network traffic incoming | for source for 2 hours. |
| and/or outgoing for a given time. | |

The control station 104 is initially configured with a set of enforcement rules that define which enforcement orders are to be given. These rules may be based on the frequency of infractions (electronic notices) a certain distribution node receives, on the nature of the infraction or on the distribution service being used. The enforcement rules (as well as the enforcement orders) should be determined by the operator of the system based on policy considerations. For example, a set of enforcement rules and orders could be chosen if it curbs illegal traffic without unduly impacting users. An exemplary enforcement rule may state that a user shall be blocked from accessing a given distribution system for 12 hour after the first time he/she uses it for illegal or undesirable purposes, for 24 hours after the second time, and be blocked from all network access forever on the third infraction.

Examples of enforcement rules and orders are given in a natural language in order to facilitate better understanding, but in the preferred embodiments these orders and rules may exist in a predetermined data format.

Once an enforcement order is chosen, the control station 104 sends the enforcement order, accompanied by the identification (usually IP address) of the offending party to the network data center 106. The network data center 106 is an existing computer networking system that is usually operated by ISPs. It is the computer hardware and software that links an ISP's client base to the Internet. In FIG. 1, the network data center 106 serves a plurality of users 108, one of which is the offending distribution node 105. In the usual network configuration all users 108 have access to the network 102 solely through the network data center 106. Thus, the network data center is in a unique position to control each user's access to the network 102.

While the network data center 106 is an existing system, it must be modified for the purposes of the present invention by adding a traffic controller 107. The traffic controller 107 is a device or a software program that is integrated within the network data center 106, which receives enforcement orders and identifications from the control station 104 and executes them by limiting the network access of the nodes that correspond to the identifications (i.e., distribution node 105) in the way defined by the enforcement orders.

The other remote nodes 103 are other computing devices that are connected to the network 102. They may include, but are not limited to, user computers, Internet servers, and other network servers. They are shown as part of the overall network environment.

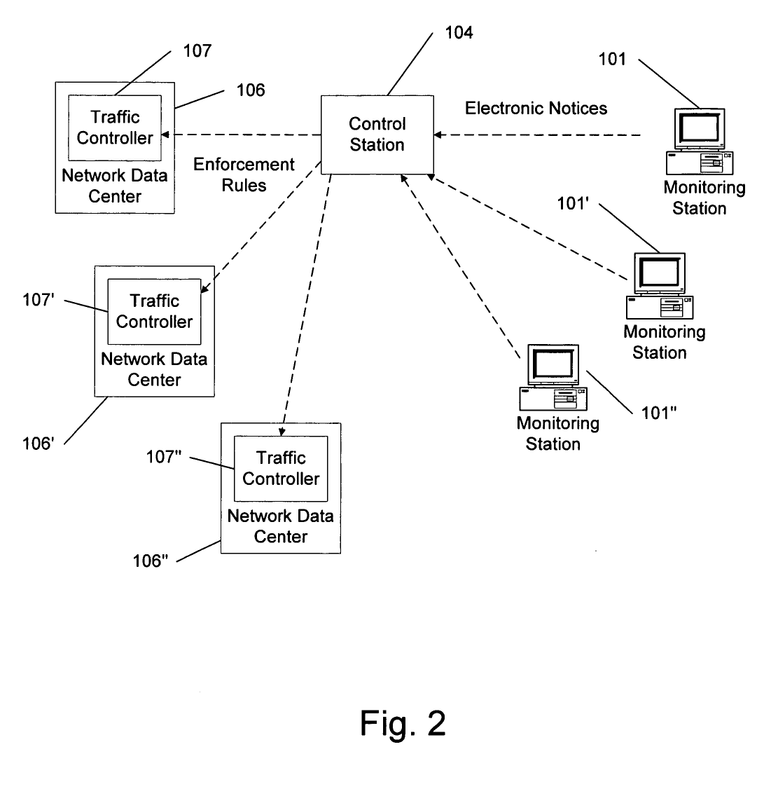

The embodiment shown in FIG. 1 is sufficient to monitor and control traffic coming from the nodes (or users) that are connected to the network 102 through a single network data center 106. The present invention may also be adapted to be used in conjunction with more than one network data center. Such an adaptation is shown in FIG. 2. In the embodiment shown in FIG. 2, there are multiple network data centers 106, 106′ and 106″, each having a corresponding traffic controller 107, 107′ and 107″. In this embodiment, the control station 104 is able to map the IP address of a user to a particular network data center. Thus, for example, the control station 104 is able to determine that a user with an IP address X is connected to network data center 106′. In this embodiment, when the control station receives an electronic notice, it examines the IP address that accompanies the notice and determines which network data center is associated with the IP address of the user. Once this determination is made, the control station generates an enforcement order in the way described above, and sends the enforcement order to the corresponding network data center.

The embodiment shown in FIG. 2 also includes multiple monitoring stations 101, 101′, and 101″. There are several reasons why multiple monitoring stations may be used. Monitoring stations may be configured so that each monitoring station monitors only one distribution channel. Thus, multiple monitoring stations are used to monitor many distribution channels. Alternatively, or in addition, the present invention may be set up so different content owners may each create their own monitoring station in order to more effectively monitor their own content, and to avoid disclosing possible trade secrets associated with content identification methods. Multiple monitoring stations operate in the same manner as the single monitoring station of the embodiment shown in FIG. 1.

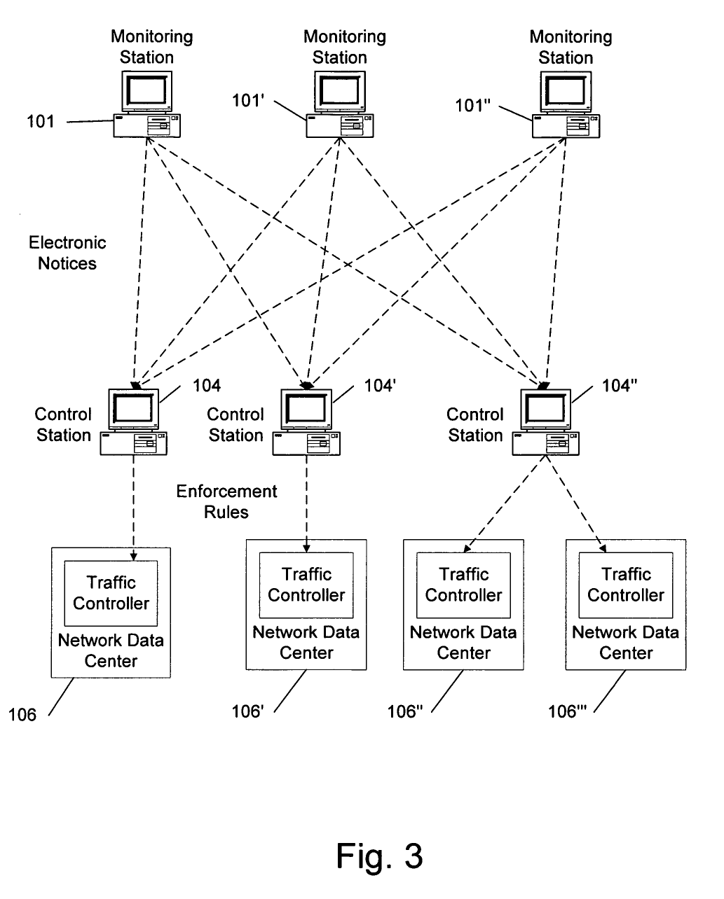

FIG. 3 shows an embodiment where multiple control stations 104, 104′ and 104″ are used. Each control station is associated with one or more network data centers 106, 106′, 106″ and 106′″. Multiple control stations may be used if the present invention is meant to cover the clients of multiple ISPs but each ISP insists on having the control station on its premises for security purposes. It may also be the case that large distances make it more efficient to use multiple control stations. In this embodiment, each monitoring station 101, 101′ and 101″ is able to map the IP address of an offending user to a specific control station. Thus, once the monitoring station identifies a target file, it maps the IP address of the source of that target file to a specific control station and sends the electronic notice to that control station. The control stations themselves operate in a way similar to the control station of the embodiment shown in FIG. 2.

The monitoring station, control station and traffic controller are implemented on computing devices having a processor and computer memory, readable by the processor. However, they need not be implemented on separate devices. As is recognizable by the person familiar with the art, the monitoring station, control station and traffic controller can all be implemented on a single computing device.

While the foregoing description and drawings represent illustrative embodiments of the present invention, it will be understood that various changes and modifications may be made without departing from the spirit and scope of the present invention.

1. A method for monitoring and controlling communications on a computer network, the computer network having a distribution node, comprising the steps of:
establishing a connection between a monitoring station and the computer network;
automatically retrieving a likely target file from the distribution node; and
automatically determining whether the likely target file contains illegal or undesirable content.

2. The method of claim 1, further comprising the step of automatically saving identification information for the distribution node.

3. The method of claim 2, wherein in the event that the automatically determining step establishes that the likely target file does contain illegal or undesirable content, the method further comprises the steps of:
automatically choosing an enforcement order; and
automatically executing the enforcement order against the distribution node.

4. The method of claim 3, wherein the enforcement order is an order to limit the network access of the distribution node.

5. The method of claim 3, wherein the step of automatically choosing an enforcement order comprises the steps of:
creating an electronic notice, the electronic notice comprising an identifier of the distribution node; and
using an enforcement rule to derive an enforcement order from the electronic notice.

6. The method of claim 1, wherein the automatically determining step further comprises the steps of:
automatically deriving a fingerprint from the likely target file;
automatically comparing the fingerprint with one or more previously stored fingerprints corresponding to one or more target files.

7. The method of claim 1, wherein the automatically determining step further comprises the step of:
automatically extracting a watermark from the likely target file.

8. The method of claim 1, wherein the automatically retrieving step, further includes the step of automatically identifying the likely target file.

9. The method of claim 8, wherein the step of automatically identifying a likely target file is performed based on one or more criteria chosen form the group of file name, file size, a hash value derived from the file, existing description of file contents, and metadata associated with the file.

10. A method for monitoring and controlling communications on a computer network, the computer network having a distribution node, comprising the steps of:
establishing a connection between a monitoring station and the computer network;
automatically retrieving a likely target file from the distribution node;
automatically determining whether the likely target file contains illegal or undesirable content;
automatically saving identification information for the distribution node;
automatically choosing an enforcement order; and
automatically executing the enforcement order against the distribution node.

11. A system for monitoring and control of communications over a computer network, comprising:
a plurality of network nodes;
a traffic controller, connected to the plurality of network nodes, and a computer network, and controlling a plurality of connections between the network nodes and the computer network;
a control station connected to the traffic controller; and
a monitoring station connected to the control station and the computer network,
wherein the monitoring station monitors whether illegal or undesirable content is being offered on the computer network, and in the event that such content is being offered sends electronic notices to the control station, the control station selects an enforcement order and the traffic controller executes the enforcement order by limiting access to the computer network from one or more of the plurality of network nodes.

12. The system of claim 11, wherein the monitoring station comprises a stored list of identifiers of illegal or undesirable content.

13. The system of claim 12, wherein the identifiers are fingerprints.

14. The system of claim 11, wherein the control station comprises a stored list of enforcement rules, and is configured to use the enforcement rules to derive enforcement orders from electronic notices.

15. The system of claim 11, wherein a plurality of traffic controllers and a plurality of monitoring stations are connected to the control station, and the control station chooses a destination traffic controller and sends the enforcement order to the destination traffic controller.

16. The system of claim 11, wherein a plurality of control stations are each connected to every one of the plurality of monitoring stations.

17. A device for monitoring and controlling network communications among several nodes on a computer network one of the nodes being a distribution node, comprising:
a processor; and
a memory storing processing instructions for controlling the processor, the processor operative with the processing instructions to:
establish a connection between the device and the computer network;
automatically retrieve a likely target file from the distribution node;
automatically determine whether the likely target file contains illegal or undesirable content;
automatically save identification information for the distribution node;
automatically choose an enforcement order; and
automatically execute the enforcement order against the distribution node.

18. The device of claim 17, wherein the memory further stores:
a plurality of enforcement rules; and
a plurality of enforcement orders.

19. A system for monitoring and controlling network communications among several nodes on a computer network one of the nodes being a distribution node, comprising a first device, the first device comprising:
a first processor; and
a first memory storing a first set of processing instructions for controlling first the processor, the first processor operative with the first set of processing instructions to:
establish a connection between the first device and the computer network;
automatically retrieve a likely target file from the distribution node;
automatically determine whether the likely target file contains illegal or undesirable content; and
automatically save identification information for the distribution node.

20. The system of claim 19, further comprising a second device the second device comprising:
a second processor; and
a second memory storing a second set of processing instructions for controlling the second processor, the second processor operative with the second set of processing instructions to automatically choose an enforcement order.

21. The system of claim 20, further comprising a second device the second device comprising:
a third processor; and
a third memory storing a third set of processing instructions for controlling the third processor, the third processor operative with the third set of processing instructions to automatically execute the enforcement order against the distribution node.
